SEQUENCE LISTING (9 sequences)

SEQ ID NO: 1 - DNA, 24 bp, Artificial Sequence
  Synthetic Construct
tgtatccctacccatctcctggca

SEQ ID NO: 2 - DNA, 26 bp, Artificial Sequence
  Synthetic Construct
ctcattacagttgacctttggcaccc

SEQ ID NO: 3 - DNA, 24 bp, Artificial Sequence
  Synthetic Construct
ttggactaccgtgaggatggtgtg

SEQ ID NO: 4 - DNA, 24 bp, Artificial Sequence
  Synthetic Construct
tgtggatgacaagcagaagccagt

SEQ ID NO: 5 - DNA, 42 bp, Artificial Sequence
  Synthetic Construct
gctcccaatatggaacatgtatacatgttccatattgggagc

SEQ ID NO: 6 - DNA, 42 bp, Artificial Sequence
  Synthetic Construct
cctggtgaacttaaacttgttaacaagtttaagttcaccagg

SEQ ID NO: 7 - DNA, 42 bp, Artificial Sequence
  Synthetic Construct
ctggtgaacttaaacttgttataacaagtttaagttcaccag

SEQ ID NO: 8 - DNA, 42 bp, Artificial Sequence
  Synthetic Construct
gcctagtaacagtaacgagtatactcgttactgttactaggc

SEQ ID NO: 9 - DNA, 42 bp, Artificial Sequence
  Synthetic Construct
ccgaggatgaatgctgtgtttaaacacagcattcatcctcgg

(19) United States
(12) Patent Application Publication
(10) Pub. No.: US 2011/0319472 A1
(43) Pub. Date: Dec. 29, 2011

(54) METHODS OF PROGNOSTICATING AND TREATING EWING SARCOMA/PNET AND OTHER NEOPLASMS

(75) Inventors: Carlos Mackintosh Ginel, Salamanca (ES); José Luis Ordonez Garcia, Salamanca (ES); Victoria Sevillano Gonzalez, Salamanca (ES); Daniel Jose García Domínguez, Salamanca (ES); Enrique de Alava Casado, Salamanca (ES)

(73) Assignee: Centro de Investigacion del Cancer - IBMCC (USAL-CSIC), Salamanca (ES)

(21) Appl. No.: 13/166,036

(22) Filed: Jun. 22, 2011

Related U.S. Application Data
(60) Provisional application No. 61/357,678, filed on Jun. 23, 2010.

(51) Int. Cl. A61K 31/7088; C40B 30/04; C12Q 1/68; A61P 35/00

(52) U.S. Cl. 514/44 A; 506/9; 435/6.12

(57) ABSTRACT

A method of distinguishing between Ewing sarcoma patients having a good prognosis relative to those having a poor prognosis by screening for specific genomic copy number alterations (CNA) isolated and/or measuring denticleless homolog (Drosophila) (DTL) protein levels from the tumors of patients diagnosed with Ewing Sarcoma.

### CROSS REFERENCE TO RELATED APPLICATIONS

This is a non-provisional application claiming priority to U.S. Provisional Patent Application No. 61/357,678 for “Methods of Prognosticating and Treating Ewing Sarcoma/PNET and Other Neoplasms” filed on Jun. 23, 2010 in the name of Carlos Mackintosh Ginel et al., which is hereby incorporated by reference herein in its entirety.

### FIELD

The present invention generally relates to a method of prognosticating the likelihood of relapse or survival for patients diagnosed with Ewing Sarcoma/PNET by screening for specific genomic copy number alterations (CNA) isolated from the tumors of said patients. Advantageously, this prognosis determination may be performed during initial diagnosis.

### BACKGROUND

Ewing Sarcoma/PNET (ES) is an aggressive neoplasm of the bone and soft tissues mainly found in children and young adults. ES is the second most common primary malignant bone tumor in children and adolescents and it belongs to a group of neuroectodermal tumors known as Ewing sarcoma Family of Tumors (EFT). Despite continuous therapeutic improvements, 5-year survival rates are below 20% in patients with advanced disease. Haeusler, J., et al., Cancer 116, 443-450.

All EFT share specific translocations resulting in the fusion of the EWS gene on chromosome 22q12 with different ETS oncogenes on different chromosomes giving rise to oncogenic chimeric proteins (EWS-ETS); the most frequent (about 95%) is FLI1 on chromosome 11 (EWS-FLI1). These translocations are considered distinct diagnostic features of ES tumors. Delattre, O. et al., New Eng. J. Med. 331, 294-299 (1994). While efforts to characterize the molecular implications of these chimeric proteins (considered the initiating molecular event in the pathogenesis of the disease) have been carried out, few studies have evaluated the role of secondary alterations. As a consequence, currently there is no molecular classification of ES, and the only alterations known to confer poor prognosis (TP53 mutations and CDKN2A deletions) are infrequent. de Alava, E., et al., Cancer 89, 783-792 (2000); Huang, H. Y., et al., J Clin Oncol 23, 548-558 (2005); Tsuchiya, T., et al., Cancer Genet Cytogenet 120, 91-98 (2000). Likewise, while major efforts in further characterizing the genomic Copy Number of Alterations (CAN) profiling of ES have succeeded in assessing the frequency of recurrent genomic alterations (Tarkkanen, M., et al., Cancer Genet Cytogenet 114, 35-41 (1999); Knuutila, S., et al., Cancer Genet Cytogenet 100, 25-30 (1998); Kullendorff, C. M., et al., Med Pediatr Oncol 32, 79-83 (1999); Brisset, S., et al., Cancer Genet Cytogenet 130, 57-61 (2001); Ozaki, T., et al., Genes Chromosomes Cancer 32, 164-171 (2001); Hattinger, C. M., et al., Br J Cancer 86, 1763-1769 (2002); Roberts, P., et al., Genes Chromosomes Cancer 47, 207-220 (2008)), few have integrated genomic and transcriptomic profiles or gained in-depth information about their correlation with clinical parameters. Ferreira, B. I., et al., Oncogene 27, 2084-2090 (2008); Savola, S., et al., BMC Cancer 9, 17 (2009).

Both the primary site of the tumor, and the initial response to therapy (assessed histologically as the degree of tumor necrosis following surgery), have become accepted valid prognostic factors in localized tumors. That said, current clinical and biological characteristics fail to accurately classify ES patients according to their clinical behavior, and it is therefore essential to search for novel reliable prognostic parameters, specifically at diagnosis.

Since chimeric proteins are present in virtually all the ES tumors and because the different EWS-ETS combinations share a similar impact on the clinical outcome (Ordoñez, J. L., et al., Cancer Res 69, 7140-7150 (2009)), secondary molecular features could act as the driver of genetic alterations for a subset of ES patients, and could have potential as molecular targets for specific therapies.

### SUMMARY

It has now been found that it is possible to distinguish between ES patients having a good prognosis and those having a poor prognosis by screening for specific genomic copy number alterations (CNA) isolated from the tumors of said patients. Furthermore, it has been found that this prognosis determination may be performed very early on, for example, during initial diagnosis.

In one aspect, a method of identifying in the neoplasm(s) from a subject any of the following molecular features with prognostic value, said method comprising:
(a) obtaining a neoplastic sample from said subject; and
(b) (i) screening said neoplastic sample for genomic copy number alterations (CNA) and/or the expression levels of the 1qGSig genes, wherein neoplastic samples having 1q gain and/or the 1qGSig are statistically associated with poor prognosis for said subject;
   (ii) screening said neoplastic sample for genomic copy number alterations (CNA), wherein neoplastic samples not harbouring CNA are statistically associated with good prognosis for said subject; and/or
   (iii) measuring DTL expression levels (RNA and protein) in said neoplastic sample, wherein neoplastic samples having (i) a 1q gain and/or (ii) statistically higher amounts of DTL relative to a control are statistically associated with poor prognosis for said subject.

In another aspect, a method of inhibiting tumor growth in a subject diagnosed with a neoplasm displaying overexpressed DTL protein levels is described, said method comprising inhibiting the expression of a DTL gene by introducing an interfering nucleotide sequence that targets at least a fragment of a polynucleotide sequence of the DTL gene and silences the gene. Preferably, the neoplasm is Ewing sarcoma.

Other aspects, features and embodiments of the invention will be more fully apparent from the ensuing disclosure and appended claims.

### BRIEF DESCRIPTION OF THE DRAWINGS

FIG. 1(a) The microdeletion of CDKN2A in the STAET2.1 cell line is shown, demonstrating that the BAC library used in the aCGH study is able to detect this alteration.

FIG. 1 (b) The microdeletion in 16q was detected in the SKES1 cell line by the BAC-microarray and was confirmed and accurately delimited by the SNP microarrays, restricting its size to 1.04 Megabases (Mb). Candidate genes inside the region are shown. The region displayed corresponds to the red-squared portion on the chr 16 ideogram. (BR=Breakpoint Region).

FIG. 2(a,b) CNA impact on ES patient survival by Kaplan-Meier log rank tests: PGA tightly correlates with OS (a), and patients with 1q gain ES had a marked poor OS (b). Left: data from 66 patients stratified by the 1q copy number status. Right: data from 38 patients stratified by the 1qGSig.

FIG. 2(c) Unsupervised hierarchical clustering using the whole transcriptome succeeded inidentifying a cluster that includes 1qGT and 1qGSig tumors (items squared in yellow) (1qGSig T).

FIG. 2(d) GSEA (left) and Ingenuity (right) plots showing the overrepresentation of gene sets and pathways involved in cell cycle control distinctive of the 1qGSig tumors.

FIG. 3.(a) Box plot summarizing the result from the Real Time RT-qPCR validation of the DTL overexpression found using expression microarrays was performed in a 14-tumor sample subset, including seven 1qGSig tumors (Mann-Whitney U, p<0.005; 1qGSig: 1qGSig tumors; Non 1qGSig: Non-1qGSig tumors).

FIG. 3(b) Western-blot validation of the efficiency and specificity of 5 different shRNA constructions in decreasing DTL protein levels and demonstrating a subsequent induction of apoptosis by cleaved-caspase 3 blot.

FIG. 3(c) Fluorescence microscopic image showing a cellular population from an ES cell line efficiently transduced with pLKO.1-GFPturbo construction.

FIG. 3(d) Flow cytometry analysis of the RM82 cell line transduced with a shRNA construction against DTL showing (left) a cell cycle arrest in G0/G1 and (right) an increase in apoptosis. Blue colour is used to depict RM82 control population (transduced with the pLKO.1-NTC) while red is used for the DTL-silenced population. The populations compared contain the same number of cells.

FIG. 3(e) Assessment of the transduction efficiency of a population of TC32-cell line transduced with the pLKO.1-GFP construction, by means of flow cytometry fluorescence histogram wherein the peak to the right represents a control population (transduced with the pLKO.1-non-tageting control) and the peak to the left represents the population transduced with the pLKO.1-GFP construction). X-axis values are fluorescence intensity in log-scale.

FIG. 4(a) Western blot of progressively silenced ES cell lines, RM82 (top) and TC32 (bottom). Silencing is expressed as the percentage of undiluted lentiviral supernatant used. Dark triangles represent the decrease in silencing from left to right. The STAET2.1 (abbreviated ET2.1 in the image) cell line is included (top panel) as a reference, since it is a non-overexpresser of DTL.

FIG. 4(b) Analysis of changes in cell cycle and apoptosis induced by the DTL-progressive silencing showing (top) a progressive decrease in the S-phase cell population (lines correspond to a lineal regression, R² is shown), and (bottom) the progressive increase in apoptosis, higher in TC32 than in RM82. Apoptosis is depicted as the percentage of the dying-dead population, ranging from early apoptosis (annexin V staining alone) to necrosis (only propidium iodide staining).

FIG. 4(c) The studies done in mice confirmed the effect of DTL-silencing on cellular proliferation in vivo. Left: representative Western-blot showing the progressive silencing of DTL; center: Box plots displaying log 2-fold increases in tumor weight of each control (NTC) relative to the DTL-silenced tumor (shDTL) from the same mouse, values in Y-axis are represented in non-logarithmic scale; right: example images from a xenografted animal and a pair of tumors from each cell line belonging to a same animal (NTC and shDTL tumors).

FIG. 4(d) DTL silencing-induced decrease in proliferation according to the degree of silencing. Y-axis: MTT values are referred to as the percentage of 570 nm-absorbance of each condition relative to the non-targeting control absorbance for each time-point.

FIG. 5 Expression microarray levels of TP53, CDKN1A and CDKN1B probesets in DTL-silenced ES cell lines or the same cell lines treated with the pLKO.1-non-targeting control. Y-axis shows the fluorescence signal after normalizing the data with the RMA algorithm.

FIG. 6(a) Western-blot of three ES cell lines reveals different expression patterns of p21, p27 and p53 induced by DTL-silencing. Increases in p53 and/or p21 and/or p27 were detected in all cell lines. A decrease in CDT1 was detected only in RM82. (NTC: non-targeting control; shDTL: DTL silenced using sh280+sh2495 constructions).

FIG. 6(b) Box plot summarizing the evaluation of the Ki-67 IHC on a TMA from ES tumors according to their 1qGSig status (top) and the corresponding contingency table (bottom). Y-axis units correspond to the Ki-67 score. As a result of these observations, it was concluded that 1qGSig-tumors have higher proliferation rates (Kendal-Tau, p=0.008, Mann-Whitney U, p=0.02).

FIG. 6(c) Two example pictures of Ki-67-staining on ES tumor samples. Top: picture showing negative staining in nuclei of tumor cells (scored 0), Bottom: positive staining in nuclei of tumor cells (scored 3).

FIG. 7 GSEA plot showing the DTL silencing-induced profile enrichment in the most significant genes from the 1qGSig-related transcriptomic profile.

FIG. 8 Additional tumor samples from the 38-tumor set that shared the 1qGSig. Unsupervised hierarchical clustering led to the identification of additional ES tumors (n=3) sharing the 1qGSig, located in the leftmost cluster (separated by the yellow line). 1qGT: tumors with known 1q gain genomic status; 1qNT: tumors with known 1q normal genomic status; 1qGSig T: tumors found to share the 1q local signature characteristic of 1qG tumors; non-1qGSig T: tumors not sharing the 1q local signature characteristic of 1qG tumors.

### DETAILED DESCRIPTION AND PREFERRED EMBODIMENTS

As mentioned, hereinabove, ES is the second most common primary malignant bone tumor in children and adolescents. In spite of advances in multimodal therapy, about 50% of patients eventually relapse, even after 5 years or more. Currently accepted clinical prognostic factors fail to classify ES patients' risk to relapse at diagnosis.

It has now been found that it is possible to distinguish between ES patients having a good prognosis and those having a poor prognosis by screening for specific genomic copy number alterations (CNA) isolated from the tumors of said patients. Furthermore, it has been found that this prognosis determination may be performed very early on, for example, during initial diagnosis. Specifically, the present invention generally relates to the identification of subsets of neoplasm in a subject based on molecular features, wherein the subset may be indicative of poor prognosis and low survival rates. More specifically, the present invention generally relates to a method of identifying a subset of Ewing Sarcoma in a subject, wherein subset classification is associated with poor prognosis.

The term “good prognosis” is used herein to indicate that the patients are not expected to show ES-related signs, symptoms or evidence for a period of time compatible with the usual clinical meaning of the term. In many cases, this may be taken to mean that the patient is expected to be free from ES-related symptoms for at least five years from assessment. In other words, good prognosis includes absence of relapse or a disease-free survival for the subject. The term “poor prognosis” is similarly used to indicate that the patients are expected to relapse during treatment or within the first few years following treatment.

As defined herein, “neoplasms” are abnormal masses of tissue as a result of abnormal proliferation of cells. Neoplasms may be benign, pre-malignant or malignant. It is understood by the skilled artisan that the term tumor is synonymous with a neoplasm that has formed a lump. Neoplasms include, but are not limited to, Ewing sarcoma, lung cancer, prostate cancer, colorectal cancer, thyroid cancer, renal cancer, adrenal cancer, liver cancer, pancreatic cancer, breast cancer, Wilms tumor, myeloma, medulloblastoma, neuroblastoma, hepatic carcinoma, and central and peripheral nervous system cancer. Hing, S., et al., Am J Pathol 158, 393-398 (2001); Kjellman, P., et al., Genes Chromosomes Cancer 32, 43-49 (2001); Lo, K. C., et al., Clin Cancer Res 13, 7022-7028 (2007); Pezzolo, A., et al., Neuro. Oncol. 11(2), 192-200 (2009); Balcarkova, J., et al., Cancer Genet. Cytogenet. 192, 68-72 (2009).

Although the traditional opinion is that EWS-ETS is not only necessary but also sufficient for ES development (The International HapMap Project, Nature 426, 789-796 (2003)), recent evidences point to other genetic alterations being required. Indeed, several authors have found that exogenous expression of the ES chimeric fusions fails to induce a higher proliferation rate and to transform cells even in permissive cellular backgrounds. Lessnick, S. L., et al., Cancer Cell 1, 393-401 (2002); Riggi, N., et al., Cancer Res 68, 2176-2185 (2008).

In the present invention, a large number of ES cases were studied, with the aim of identifying subsets of patients with poor prognosis based on their molecular features. Using Copy Number Alterations (CNA) profiling it was surprisingly discovered that 1q gain, present in 31% of the samples tested, is markedly associated with relapse and poor survival of patients diagnosed with ES, wherein the 1q gain is linked to a profile of cell cycle deregulation in a 105-ES tumor set. Moreover, 1q gain tumors (1qGT) showed higher proliferation rates. This cooperative effort defines a subset of ES with differential prognosis and may afford a rationale for new therapies.

Further, it was surprisingly discovered that the candidate gene DTL (denticleless homolog Drosophila), the main gene overexpressed in 1q gain ES tumors, may be a major player for this gene in the molecular deregulation distinctive of this ES subtype.

Accordingly, in one aspect, a method of identifying a subset of neoplasm in a subject is described, said method comprising:
(a) obtaining a neoplastic sample from said subject; and
(b) screening said neoplastic sample for genomic copy number alterations (CNA) and/or 1qGSig;
   wherein neoplasmic samples having a 1q gain and/or the 1qGSig are statistically associated with poor prognosis for said subject. Preferably, the neoplasm is Ewing sarcoma.

Accordingly, in one aspect, a method of identifying a subset of neoplasm in a subject is described, said method comprising:
(a) obtaining a neoplastic sample from said subject;
(b) screening said neoplastic sample for genomic copy number alterations (CNA) and 1qGSig;
(c) measuring DTL expression levels (RNA and protein) in said neoplastic sample, wherein neoplastic samples having (i) a 1q gain and (ii) statistically higher amounts of DTL relative to a control are statistically associated with poor prognosis for said subject. Preferably, the neoplasm is Ewing sarcoma.

In another aspect, a method of identifying a subset of neoplasm in a subject is described, said method comprising:
(a) obtaining a neoplasmic sample from said subject; and
(b) measuring DTL expression levels in said neoplasmic sample,
   wherein neoplastic samples having statistically higher amounts of DTL relative to a control are statistically associated with poor prognosis for said subject. Preferably, the neoplasm is Ewing sarcoma.

In another aspect, a method of inhibiting tumor growth in a subject diagnosed with a neoplasm displaying overexpressed DTL protein levels is described, said method comprising inhibiting the expression of the DTL gene by introducing an interfering nucleotide sequence that targets at least a fragment of the polynucleotide sequence of the DTL gene and silences the gene. The interfering polynucleotide sequence may include, but is not limited to, antisense DNA or RNA, interfering double-stranded (siRNA) and single-stranded micro RNA (miRNA), ribozymes, chimeric sequences, or derivatives of these groups. Preferably, the neoplasm is Ewing Sarcoma.

Genomic copy number alterations can be detected using techniques well known to those skilled in the art, for example, using oligo-based array comparative genomic hybridization techniques (arrayCGH, aCGH).

Similarly, the findings discussed herein could also contribute to understand the biology of other neoplasms in which 1q gain has also been related to an adverse outcome, since the mechanisms involved could be similar to those described here. Hing, S., et al., Am J Pathol 158, 393-398 (2001), Kjellman, P., et al., Genes Chromosomes Cancer 32, 43-49 (2001), Lo, K. C., et al., Clin Cancer Res 13, 7022-7028 (2007), Pezzolo, A., et al., Neuro Oncol (2008), Balcarkova, J., et al., Cancer Genet Cytogenet 192, 68-72 (2009). Regarding the DTL contribution to cancer, this is a emerging area of research since, despite its evident potential as an oncogene, few works have related it to carcinogenesis. Pan, H. W., et al., Cell Cycle 5, 2676-2687 (2006), Ueki, T., et al., Oncogene 27, 5672-5683 (2008).

Accordingly, the gain of 1q defines a subset of ES patients with poor survival, and that subsequent overexpression of DTL, a gene involved in protein ubiquitination, is able to deregulate the cell cycle. Both molecular findings are potentially useful as prognostic biomarkers.

Moreover, the absence of CNA defines a subset of said ES patients with outstandingly good prognosis.

The features and advantages of the invention are more fully illustrated by the following non-limiting examples, wherein all parts and percentages are by weight, unless otherwise expressly stated.

### Cell Lines

ES cell lines used were A4573, A673, CADO-ES, CHP-100, EW3, RDES, RM82, SKES1, SKNMC, STAET1, STAET2.1, STAET10, TC71, VH64 and WE68 belong to the EuroBoNet cell line panel that is maintained, checked and characterized by Ottaviano et al. in Heinrich-Heine University at Düsseldorf. Ottaviano, L., et al., Genes Chromosomes Cancer 49, 40-51 (2010). TTC466 and TC32 came from other collections and have been previously described. Szuhai, K., et al., Cancer Genet Cytogenet 166, 173-179 (2006).

Array Comparative Genomic Hybridization (aCGH)

Whole-genome BAC-aCGH studies were performed using the Sanger 1 Mb clone set. BAC DNA was extracted, amplified by DOP and aminolinking-PCR, and triplicate aliquots were spotted onto Codelink slides (Amersham, GE).

Tumor and reference DNA (an equimolar DNA pool from 100 healthy donors, obtained from the Spanish National DNA Bank after approval from its External Ethics Committee) was Cy5/Cy3-dCTP-labelled (Amersham, GE) using a non-commercial Random Priming kit composed of random octamers dissolved in Exo-Minus Klenow buffer (Eppicentre), a dNTPs mix depleted in dCTP (Eppendorf), and Exo-Minus Klenow enzyme (Eppicentre). Labelled DNA was purified through Illustra G-50 Microspin Columns (Amersham, GE), mixed and then precipitated along with Cot Human DNA (Roche). Hybridization was performed for 48 hours at 42° C. and excess probe was removed.

The microarray data (BAC-microarrays, SNP microarrays and expression microarrays) have been deposited in NCBI's Gene Expression Omnibus (GEO; http://www.ncbi.nlm.nih.gov/geo/) and are accessible through GEO Series accession number GSE20368.

### Image Acquisition and Data Analysis of BAC-microarrays

Data was acquired using an Axon 4000B scanner and GenePix software (Axon Laboratories). Normalization was carried out with GenePix software using the mean of the median of ratios of all the autosomal features in the array and excluding those removed by the quality-flagging scripts. In those cases in which the described process of normalization was clearly unable to correctly center the aCGH log 2ratio plot, the array was re-normalized using the median log 2value from unaltered regions, taking as a reference the values from the X and Y chromosome features. Gpr files were subsequently processed with the Bioconductor (Gentleman, R. C., et al., Genome Biol 5, R80 (2004)) packages snapCGH (Smith, M. L., et al., snapCGH: Segmentation, Normalization and Processing of aCGH Data Users' Guide. Bioconductor (2006)) and CGHcall (van de Wiel, M. A., et al., Bioinformatics 23, 892-894 (2007)), incorporating scripts for removing SD>0.2 and GenePix-flagged spots. CGHcall was applied in order to reduce the data complexity by first applying a segmentation algorithm, DNAcopy (Olshen, A. B., et al., Biostatistics 5, 557-572 (2004)), and next performing the assignation of a copy number state to every segment according to an algorithm that takes into account the user's input of cellularity percentage per sample. CGHregions (Mark A. van de Wiel, et al., Cancer informatics 3, 9 (2007)) and WECCA (Van Wieringen, W. N., et al., Biostatistics 9, 484-500 (2008)) scripts were applied for clustering.

### Data Analysis of Expression and SNP Microarrays:

Analysis of expression microarray data was performed in R with the affy (Irizarry, R. A., et al., Nucleic Acids Res 31, e15 (2003)) and siggenes (Tusher, V. G., et al., Proc Natl Acad Sci USA 98, 5116-5121 (2001)) bioconductor packages. Briefly, RMA (Irizarry et al.) was applied for background correction and normalization while SAM algorithm (Tusher et al.) was used for differential expression. Clustering analyses were conducted with TIGR MeV4 software (Saeed, A. I., et al., Biotechniques 34, 374-378 (2003)).

GSEA software, version 2.0, was applied for gene set enrichment analysis (Subramanian, A., et al., Proc Natl Acad Sci USA 102, 15545-15550 (2005)). This analysis statistically assesses the overrepresentation of a gene set (from a public database or local user's generated gene sets) in the user's differentially expressed gene list ranked by signal to noise (most consistently differentially expressed genes placed in the list's first positions) or by other selectable ranking criteria. The degree of enrichment is reflected in the Enrichment Score. In the experiments described herein, the rank-ordered list corresponds to those genes differentially expressed between 1qGSig tumors and non-1qGSig tumors. A False Discovery rate (FDR) q-value is also calculated as the result of a 1000 permutations of the items in both phenotype classes considered. FDR's under 25% are considered to be the only ones that can be reliably used as sources of further biological hypothesis. The Enrichment Score is combined with the q-value to obtain the most representative value of the analysis: the Normalized Enrichment Score (NES). Those gene sets having high ES and low FDR will have the most significant NES values and will provide the most reliable coincidences with the user's data.

Gene sets are lists of genes arranged along with a criterion that varies depending on the MSigDB gene set collection (databases from the Broad Institute) selected:
C1 gene set collection: genes grouped along with a common genomic position, sorted by chromosome bands.
C2 gene set collection: genes arranged according to experimental evidence, information from differential expression studies performed in hundreds of publications from many different areas of research. It also includes gene sets from on-line pathways databases.
C3 gene set collection: genes arranged along with a shared regulatory element in the promoter or the 3′-UTR: common targets of transcription factors and miRNAs.
C4 gene set collection: genes grouped by classic Gene Ontology (GO) terms.

Comparison of the differentially expressed gene list from the 1 qGSig tumors vs. the non-1qGSig tumors with the differentially expressed gene list from the DTL-silenced cell lines was performed according to the following procedures:
1. RMA-normalized tables were firstly summarized so that genes with several probesets were collapsed into one, selecting the probeset with maximum signal.
2. SAM was applied to the summarized data (unpaired SAM for the 38-tumor data and paired SAM for the DTL silencing data).
3. A gmt file was created with 1 gene set (320 genes) corresponding to the SAM result from the tumors, with a FDR threshold of 0.001; delta 2.8.
4. The complete paired-SAM result from DTL silencing was sorted by d-value and input into GSEA “pre-ranked Gene List” analysis with 1000 permutations, selecting locally created gmts and the entire C2 MsibDB gene set collection (v2.5) (1892 gene sets). Permutations where done by shuffling gene set for gene set.

Analysis of Ingenuity Pathways (Ingenuity® Systems, www.ingenuity.com) was used for the integration of differentially expressed genes in pathways and validation of the GSEA results.

BioGPS (Wu, C., et al., Genome Biol 10, R130 (2009)) was used to check mRNA-expression levels of DTL in human tissues in the HG_U133A/GNF1H and GNF1M Gene Atlas data sets (Su, A. I., et al., Proc Natl Acad Sci USA 101, 6062-6067 (2004)).

SNP-arrays were analyzed with GTYPE 4.1 and CNAT 4.0 software (Affymetrix). The CEL files were processed to study copy number alterations, smoothing=0.1, using the Mapping 500K Sample Data Set which includes CEL files from 48 samples (96 hybridizations, considering both Nsp and Sty arrays) belonging to the International Hap Map project (Thorisson, G. A., et al., Genome Res 15, 1592-1593 (2005)), as healthy references.

Expression and SNP Microarrays

Total RNA from DTL-silenced cell lines was extracted with Trizol (Invitrogen) and hybridized with the GeneChip® Human Genome U133 Plus 2.0 Array (Affymetrix) after quality assessment using an Agilent 2100 Bioanalyzer (Agilent).

DNA from the SKES1 cell line was hybridized with the GeneChip® Human Mapping 500K Array Set (Affymetrix).

Lentiviral shRNA Transduction of ES Cell Lines for Functional Validations

The MISSION shRNA collection of 5 pLKO.1-shRNA constructions (SIGMA Aldrich) was selected for assays aimed at reducing DTL overexpression. The pLKO.1-non-targeting control construction (pLKO.1-NTC) was included as a control. Constructions were transfected separately in the 293T packaging cell line along with the pMD.G and pCMV-dR8.91 vectors, following a typical lipofectamine 2000 (Invitrogen) protocol. At 48 hours after transfection, viral supernatants were collected, fresh medium added, passed through a 0.44 μm-polysulfonate filter (PALL), polybrene-added to a final 8 μg/ml concentration (SIGMA Aldrich) and used to transduce target ES cell lines.

For assessing the transduction efficiency, TC32 was transduced with the pLKO.1-turboGFP vector and was evaluated by flow cytometry after 48 hours.

For gradual silencing, sh280 and sh2495 supernatants were mixed and progressively diluted with fresh medium before being applied to target cells. After reseeding, cell cycle, apoptosis and proliferation assays were performed at several time points.

### In Vivo Functional Validation of DTL

TC32 and RM82 cell lines were transduced with a progressively diluted DTL-shRNA viral supernatant. Both cell lines were also transduced separately with the pLKO.1-NTC. 48 hours after transduction, cells were trypsinized and suspensions containing 2×10⁶ living cells in a 0.2 ml-final volume composed of RPMI medium and Matrigel Matrix (BD) in a 1:1 proportion, were injected s.c. in one flank (DTL-shRNA transduced cells) or in the other (cells transduced with the pLKO.1-NTC) of five-week old NOD/SCID female mice (Charles River). Twelve mice were used for each cell line (quadruplicates of each silencing condition). The animals were euthanized by anesthetic overdose 19 days after cell injections, and tumors were collected for histopathology analysis, and weights were measured. The study was previously approved by the Bioethics Committee of the CIC-USAL institution.

### Treatment of Patients

Follow up data was available for all patients. Time to follow up was at least five years or until relapse. Tumor material from 27 patients treated at different institutions in Germany and collected at the Heinrich Heine University Düsseldorf was analyzed. The patients were enrolled in the Euro-E.W.I.N.G. 99 trial (n=21) (Juergens, C., et al., Pediatr Blood Cancer 47, 22-29 (2006)), or in the CESS'86 (Paulussen, M., et al., J Clin Oncol 19, 1818-1829 (2001)), EICESS'92 (Paulussen, M., et al., J Clin Oncol 26, 4385-4393 (2008)), or CWS'96 (Klingebiel, T., et al., Pediatr Blood Cancer 50, 739-745 (2008)). All patients received surgical resection and/or radiotherapy for local tumor control. Regarding 40 patients from the Catholic University of Leuven, nine patients received chemotherapy according to the scheme consisted of Vincristine, Dactinomycin/Actinomycin-D and Doxorubicin/Adriamycin and were supplemented with either Ifosfamide or Cyclophosphamide. The agents used were therefore comparable to those used in the trials described above. For others, the EICESS'92 protocol (n=8) or the Euro-E.W.I.N.G. 99 protocol (n=23) was followed. All patients received surgical resection and/or radiotherapy for local tumor control, except two, due to tumor location or metastasis at diagnosis.

### DNA Extraction

Genomic DNA was extracted from frozen tumor biopsies containing at least 70% of tumor cells. Tissue samples containing less than 80% of tumor cells, as quantified by histological examination, were microdissected from unstained glass slides prior to DNA extraction. DNA was isolated using the Qiagen DNA Extraction kit (Qiagen) or the High Pure PCR Template Preparation kit (Roche) following the manufacturer's recommendations.

### SYBR Green Real Time RT-qPCR

This was performed as previously described (Martins, A. S., et al., Cancer Res 68, 6260-6270 (2008)). Primers were designed with primer3 software (Rozen, S. et al., Methods Mol Biol 132, 365-386 (2000)) using the same sequence covered by Affymetrix probesets and pre-tested for non-especific amplifications with Primer-blast (NCBI). DTL-primers: Fw-TGTATCCCTACCCATCTCCTGGCA (SEQ ID NO: 1), Rv-CTCATTACAGTTG ACCTTTGGCACCC (SEQ ID NO: 2). For ddCT normalization, the TPT1 housekeeping gene was selected with a bioinformatics approach using the expression microarray data from the 38-tumor sample set since it was the gene with the minimum expression change. TPT1 primers: Fw-TTGGACTACCGTGAGGATGGTGTG (SEQ ID NO: 3), Rv-TGTGGATGACAAG CAGAAGCCAGT (SEQ ID NO: 4).

### Apoptosis (Annexin V-Propidium Iodide Method) and Cell Cycle Assays

Both assays were carried out by flow cytometry as previously described (Abbas, T., et al. PCNA-dependent regulation of p21 ubiquitylation and degradation via the CRL4Cdt2 ubiquitin ligase complex. Genes Dev 22, 2496-2506 (2008)).

### Proliferation Assays

Cell proliferation was assessed by MTT-assays, as described elsewhere (Martins, A. S., et al., Clin Cancer Res 12, 3532-3540 (2006)).

### Western-Blot

Protein extracts in RIPA were resolved in 8% and 12% polyacrylamide gels (40 μg of protein loaded) and transferred to PVDF membrane (Bio-Rad) with a semidry transfer device (Bio-Rad). 5% Dry-milk in TBS-T was used as a blocking agent and antibodies were incubated overnight at 4° C. (1:200-1:1000 dilution). Santa Cruz BT antibodies were: cyclin D1 (sc-718), Cyclin E (sc-247), anti-p21 (sc-397), p53 (sc-126 and sc-98). Anti-p27 was from Pharmingen (554069); anti-CDT1 was from Abeam (ab70829) and anti-β-tubulin (t-5293) and anti-cyclin A (c-4710) were purchased from SIGMA Aldrich. Anti-cleaved caspase-3 was from Cell Signaling (4669) and anti-DTL antibody was a kind gift from Dr. Hung-Wei Pan and Dr. Hey-Chi Hsu (Department of Pathology, College of Medicine, National Taiwan University, Taipei, Taiwan).

### Immunohistochemistry

IHC was performed on 4 μm-thick sections of a TMA composed of 33 formalin-fixed paraffin-embedded specimens from the 38-ES tumor set. The antibody used was anti Ki-67 (MIB-1, Dako). Antigen retrieval was performed by pressure cooking in citrate buffer (pH 6.0). The LSAB detection method (Dako) was applied. All sections were evaluated independently by 2 pathologists and the score was agreed in case of discrepancy. Only nuclear staining was considered positive. Immunoreactivity was scored according to the scale: negative (0) for less than 5% of tumor cells stained, weakly positive (1) for 5% to 10% of tumor cells stained; moderately positive (2) for 10% to 50% of tumor cells stained and strongly positive (3) for more than 50% of the tumor cells stained.

### Statistical Analyses

The SPSS software, version 15.0, was applied for statistical analyses.

For analyses of the results from the in vivo studies, log 2 transformation of the ratio of weights (the control tumor weight divided by the silenced tumor weight of each animal) was used for maintaining the normal distribution of the paired data (Kolmogorov-Smirnov<0.05). Multi-factorial ANOVA discarded the existence of differences in the data due to the cell line factor and validated the effect of the shRNA treatment factor (p value=0.05). Thus, data from both cell lines were considered together for further analysis. T-test was applied to validate the difference between the mean of each shRNA treatment condition and the log 2value corresponding to the expected absence of change (H=0).

### Example 1

### CNA are Clinically Relevant in ES and 1q Gain has a Profound Impact on the Relapse and Poor Survival of a Subset of ES Patients

The tumor set used in aCGH studies corresponded to 66 primary tumors and 1 pulmonary metastasis from ES patients collected at two institutions of the EuroBoNet consortium (Catholic University of Leuven, n=40; Heinrich-Heine-University, n=27). Forty-four patients were males and 23 females, with ages from 1 to 84 years-old (median age=15), with primary tumors localized in bones (n=47) and in soft tissues (n=19). Seventeen patients were diagnosed for primary disseminated disease. The diagnosis was performed by experienced sarcoma pathologists and all tumor specimens were confirmed to harbor an EWS-ETS by FISH, using split-apart EWS probe (Vysis), or by RT-PCR. Friedrichs, N., et al., Diagn Mol Pathol 15, 83-89 (2006). Approval of the Ethics Committees involved is available from all cases and written informed consent was obtained before registration of the patients.

Correlation of classical clinical parameters (primary tumor location, size, presence of metastasis or local relapse, etc) with survival was assessed to ensure they were the usual ones for this entity. For studies correlating aCGH data with clinical parameters only primary tumors were considered.

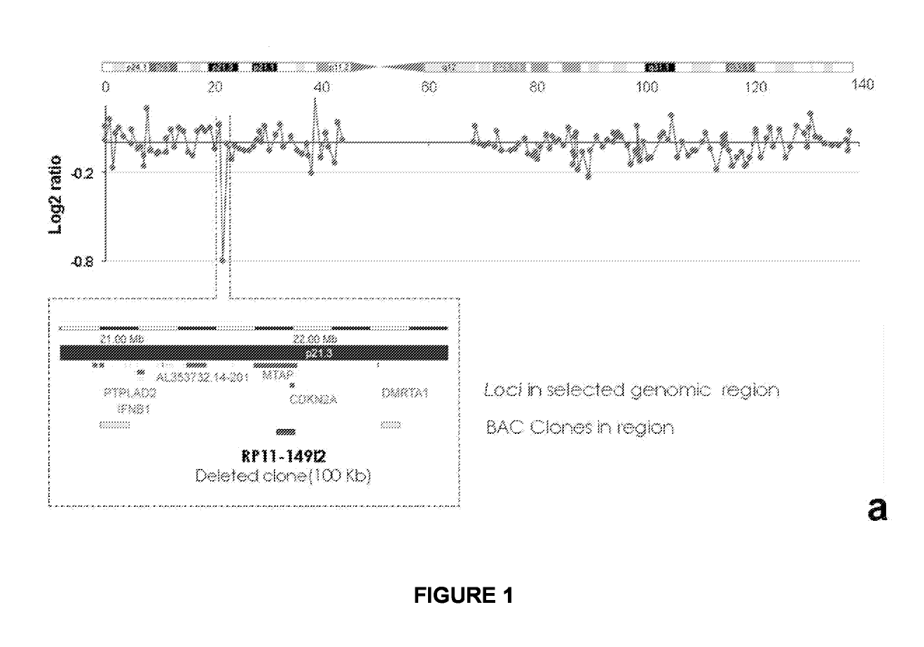

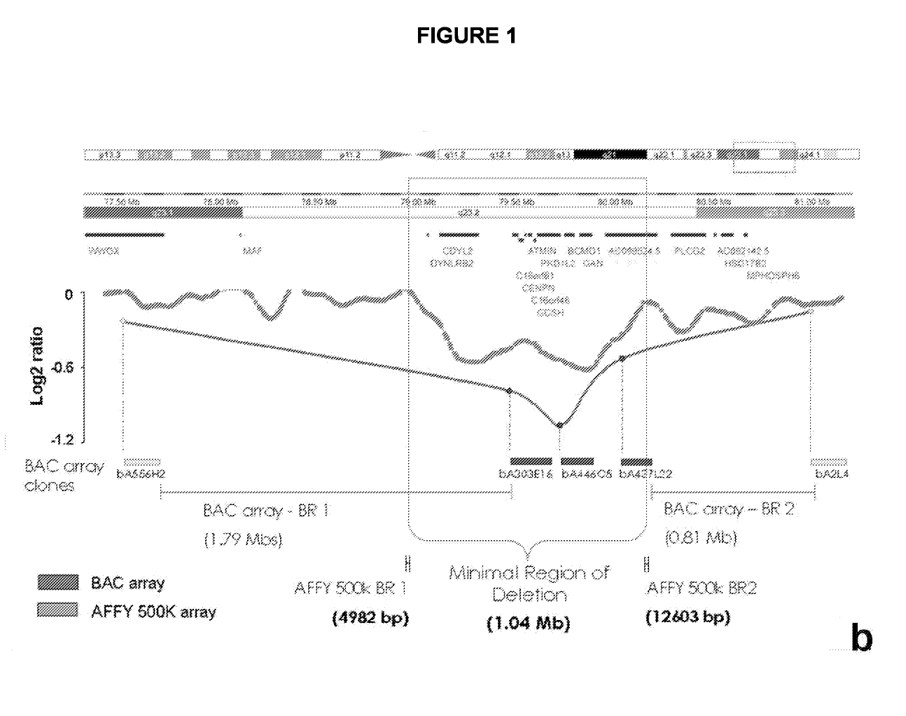

ES tumor samples from 67 untreated patients and 16 ES cell lines were screened for genomic copy number alterations (CNA) by arrayCGH (aCGH). The profile of recurrent alterations was concordant with that described in previous reports: the most frequent gains included the entire chromosome (chr) 8, 1q, and 12p, while the most frequently lost regions were located in 16q, chr 10, 9p, and 17p. Remarkably, although CDKN2A deletion was one of the most frequent CNA in cell lines (62.5%), it was detected in only 8 tumors (12%); in 5 of them as a single-BAC microdeletion (FIG. 1a). The same was the case of deletions encompassing the TP53 locus, which were frequent in the cell lines (37.5%) but uncommon in the tumor samples (13.4%).

The smallest regions of overlap (SRO) under 10 Mb in size and with a frequency above 35% were further evaluated using the aCGH data from both the tumor samples and the cell lines. Affymetrix 500K SNP microarrays (AFFY 500k) were used to accurately delimit some of these SRO, like the one in the most frequently deleted region, 16q23.2, of 1.04 Mb in size, which rendered a small collection of candidate genes (FIG. 1b).

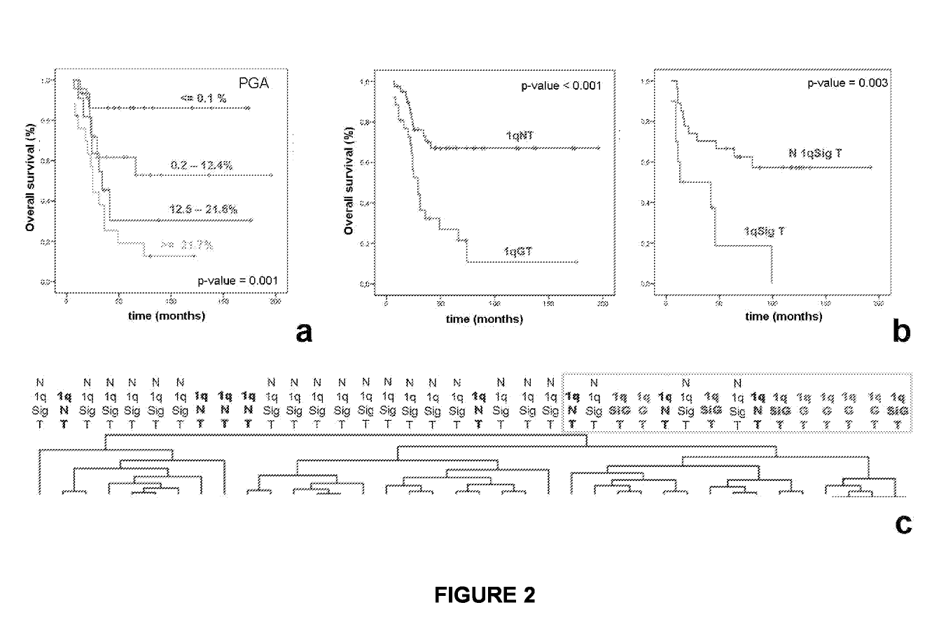

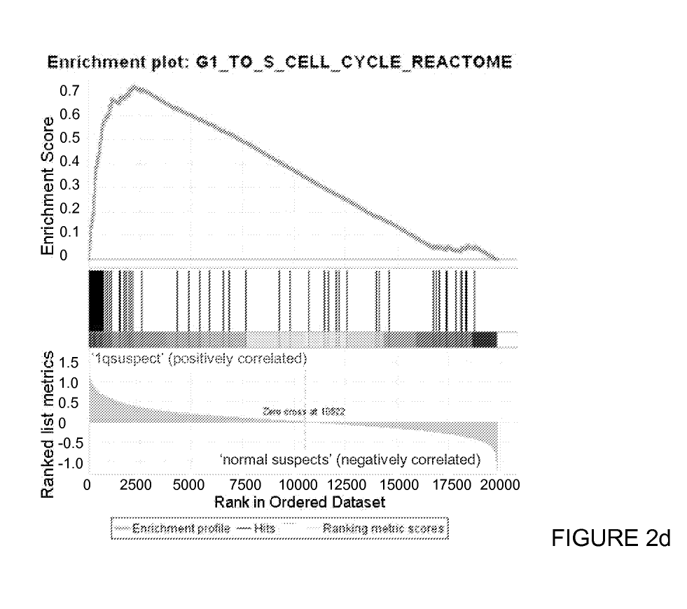

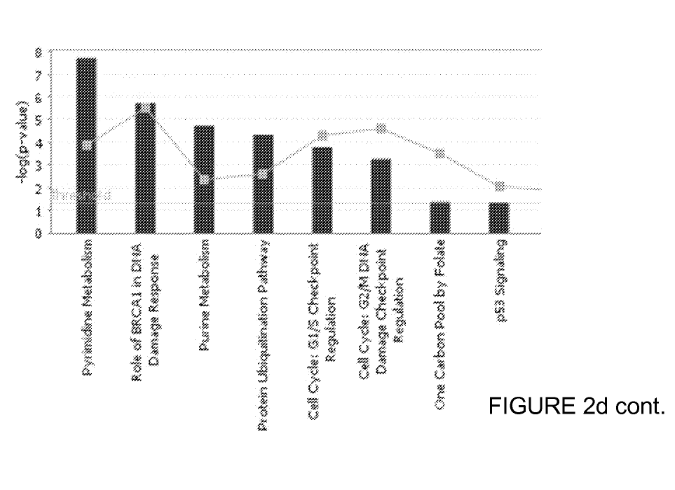

The Percentage of Genome Altered (PGA) parameter was calculated to estimate the total amount of the genome affected by CNA per sample. A program named “CGH altered genome” was written by the first author in R language (Dessau, R. B., et al., Ugeskr Laeger 170, 328-330 (2008)) in order to estimate the Percentage of Genome Altered (PGA) and the Overall Number of Copy Number Alterations (OCNA) in each sample. The program enumerates each segment produced by the DNAcopy segmentation algorithm (Olshen, A. B., et al., Biostatistics 5, 557-572 (2004)) in each sample, and determines its size (last BAC genomic position in the segment minus the first BAC position). Then, the overall number of segments altered is calculated (identified by the CGHcall algorithm; hereinafter the OCNA) as well as their overall size, and finally the fraction that each size represents in relation with the overall size of the human genome included in the BAC microarray is determined (hereinafter, the PGA parameter, expressed as a percentage). PGA was tightly correlated with poor survival in Kaplan-Meier analysis, demonstrating the impact that CNA have on ES clinical outcome (FIG. 2a). Remarkably, patients without CNA (PGA<0.01%) had an outstandingly good survival (over 85% at 5 years).

CGHtest BioC package (van de Wiel, M. A., et al., Bioinformatics 21, 3193-3194 (2005)) was used in supervised pair-wise comparisons for the screening of CNA statistically enriched in selected clinical subsets and it was also applied to detect any CNA significantly correlated with poor survival. Specifically, for the correlation of CNA with the clinical data, CGHregions, CGHPermutations2.1 and CGHlogrank2.0 scripts were used (van de Wiel, M. A., et al., Bioinformatics 21, 3193-3194 (2005)). The CGHPermutations script was set to the Wilcoxon rank-sum test for two groups (mcsteel option) and 10000 permutations. CGHlogrank was set to 10000 permutations, teststat=logrank and used either with Overall Survival (OS) or with Disease-Free Survival (DFS) data. Correlations of PGA/OCNA with survival in Kaplan-Meier analyses were done by splitting samples in six groups by 5 percentile cut-offs. If two contiguous groups showed a similar survival curve they were joined together. A complete summary of the tests performed and their results is shown in Table 1.

TABLE 1
Results from the bioinformatics screening of CNA-clinical implications
Test type: CGH Permutations

| Conditions tested | Significant | p-value/ |
|---|---|---|
| Primary tumor with osseous | NS | |
| location vs. ononsseous | | |
| Relapse (metastasis plus | 1 q gain (entire arm) | 0.01/0.046 |
| local relapse) vs. non relapse | | |
| Metastasis vs. non metastasis | NS | |
| Metastasis at diagnosis vs. | NS | |
| new metastasis | | |
| Single metastasis vs. | NS | |
| multiple metastasis | | |
| Metastasis to sites including | 8 p gain (entire arm) | 0.017/0.03 |
| lung vs. metastasis to | 8 q gain (entire arm) | 0.002/0.005 |
| sites not including lung | | |
| Patient younger than 18 vs. | 16 p loss (pter-2 Mb) | 0.03/0.057 |
| older than 18 | | |
| Patient younger than 24 vs. | 16 p loss (pter-2 Mb) | 0.026/0.066 |
| older than 24 | 8q gain (124 Mb-129 Mb | 0.003/0.06 |
| | from pter) | |
| Cell lines vs. tumors with | 3 p loss (40 Mb-75 Mb | 0.004/0.02 |
| PGA > 1% | from pter) | |
| | 9 p loss (pter- 24 Mb | 0.005/0.014 |
| | from pter) | |
| | Chr 12 gain (qter-pter) | 0.005/0.005 |
| Tumors with 1 q gain vs. | Chr 20 gain (qter-pter) | 0.002/0.002 |
| tumors with normal 1 q | Chr 16 loss (45-90) | 0.02/0.07 |

Test type: Survival Log Rank Test

| | | |
|---|---|---|
| | Chromosome decom- | p-value/ |
| Chromosome alteration | posed in Regions | FDR |

Overall Survival

| | | |
|---|---|---|
| 1 q gain | 141.4 Mb-152.8 Mb | 0.004/0.02 |
| | from pter | |
| | 154.6-180.3 | <10-3/<10-3 |
| | 183.4-195.0 | 0.001/0.016 |
| | 196.2-245.0 | 0.002/0.021 |
| | 0.2-53.4 | 0.01/0.035 |
| | from pter | |
| Chr 20 gain | 54.5-61 | 0.005/0.023 |
| | 61.5 - 62.8 | 0.003/0.02 |
| Chr 22 loss | 15.6-50 | 0.014/0.078 |

Disease-free Survival

| | | |
|---|---|---|
| 1 q gain | 141.4 Mb-152.8 Mb | 0.002/0.21 |
| | from pter | |
| | 154.6-180.3 | <10-3/<10-3 |
| | 183.4-195.0 | 0.011/0.07 |
| | 196.2-245.0 | 0.004/0.03 |
| Chr 10 loss | 1-135.5 | 0.005/0.06 |

(Mb = Megabases; NS = no significant results obtained)

Briefly, the gain of 1q, detected in 31% of the tumor samples, was the CNA with the highest impact on the clinical parameters because:

1.—It was found to be the only alteration differentially present in tumors from ES patients developing clinical relapse, either local or distant metastasis (p-value=0.01, q-value=0.046).
2.—It was the CNA displaying the closest correlation with poor survival, with both Overall Survival (OS) and Disease-Free Survival (DFS), producing the most significant Kaplan-Meier curves when using OS data (FIG. 2b, left panel).

The effect of the 1q gain on survival did not depend on a higher PGA, since a Wilcoxon Rank test of 1q-gain tumors (1qGT) and 1q-normal tumors (1qNT) was not significant for differences in PGA (considering patients with PGA>0%). Nevertheless, several alterations were detected as “partners” of the 1q gain owing to their tendency to specifically appear in 1qGT: gains of the entire chr 12 and chr 20, and loss of 16q. In any case, according to Kaplan Meier analysis no differences were found in OS between patients with a 1q gain alteration alone and patients with both a 1q gain CNA and any of its CNA-partners.

The impact of 1q gain on survival was confirmed in an independent 38-ES tumor set classified as explained below (FIG. 2b, right panel).

As expected because of the correlation between 1q gain and clinical relapse, Cox multivariate analyses showed the 1q gain parameter was not an independent prognostic factor.

### Example 2

The Gain of 1q Exerts Strong Effects on the Transcriptome and is Associated with a Pattern of Cell Cycle Deregulation in ES.

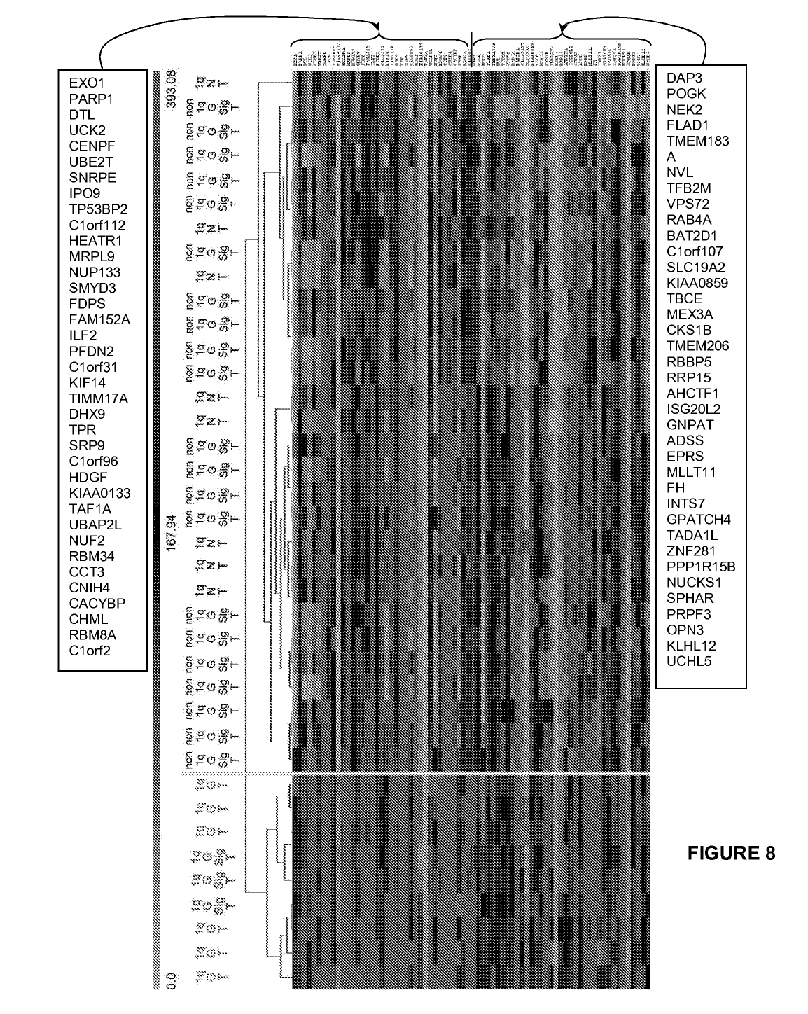

An independent 38-ES tumor set, partially profiled for CNA and with available expression microarray data, was used for assessing the changes induced by 1q gain on the transcriptome. Savola, S., et al., BMC Cancer 9, 17 (2009). A description of the tumor series (n=38) used in transcriptomic studies and in TMAs can be found elsewhere. Scotlandi, K., et al., J Clin Oncol 27, 2209-2216 (2009). The tumor set was divided in two groups according to a 1q gain expression signature (1qGSig) defined by the 74 genes located in 1q with the highest d-values and fold-change values (cut-off settled: 90^{th} percentile, d-value>4.9; fold change, R-fold, above 1.5) (the complete gene list can be found in FIG. 8), from a significance analysis of microarrays (SAM) analysis performed comparing the 1qGT and the 1qNT. 1qGSig reflects the underlying genomic 1q copy number status.

Unsupervised hierarchical clustering succeeded in distinguishing tumors according to the 1qGSig (FIG. 8), suggesting that the 1q gain exerts a strong impact on the transcriptome (FIG. 2c). It should be noted that the mismatches between the clustering and the 1qGSig classification were caused by non-1qGSig tumors (N 1qGSig T) “infiltrating” the 1q gain core cluster, which could be indicative that these non-1qGSig tumors share the 1q gain expression patterns although through different underlying molecular mechanisms.

Next, the differential expression profile from 1qGSig tumors was compared with the Gene Set Enrichment Analysis (GSEA) (Subramanian, A., et al., Proc Natl Acad Sci USA 102, 15545-15550 (2005)) gene set collections (see, Table 2, wherein datasets are divided by MSigDB gene set collections. (NES=Normalized Enrichment Score)).

TABLE 2
GSEA results from 1qGSig tumors vs Non-1qGSig tumors

| | | | NOM | FDR |
|---|---|---|---|---|
| | Size | Nes | p-val | q-val |
| C2 MSigDB Gene Set Collection | | | | |
| G1_to_s_cell_cycle_reactome | 68 | 2.193 | 0.000 | 0.001 |
| Cell_ cycle_kegg | 83 | 2.154 | 0.000 | 0.001 |
| Brentani_cell_cycle | 81 | 2.136 | 0.002 | 0.001 |
| Hsa04110_cell_cycle | 109 | 2.130 | 0.000 | 0.001 |
| C ell_cycle | 76 | 2.107 | 0.000 | 0.001 |
| Pyrimidine_ metabolism | 57 | 1.989 | 0.000 | 0.005 |
| P21_any_dn | 34 | 1.967 | 0.000 | 0.006 |
| Ren_e2fl_targets | 38 | 1.945 | 0.000 | 0.006 |
| Schumacher_myc_up | 49 | 1.938 | 0.000 | 0.006 |
| C3 MSigDB Gene Set Collection | | | | |
| V$E2F_Q4_01 | 185 | 2.128 | 0.000 | 0.002 |
| V$E2F_Q3_01 | 181 | 2.109 | 0.000 | 0.001 |
| V$E2F_Q6_01 | 182 | 2.098 | 0.000 | 0.001 |
| V$E2F1 _Q4_01 | 177 | 2.085 | 0.000 | 0.001 |
| V$E2F1DP2_01 | 185 | 2.009 | 0.002 | 0.003 |
| V$E2F1DP1_01 | 185 | 2.009 | 0.002 | 0.002 |
| V$E2F4DP2_01 | 185 | 2.009 | 0.002 | 0.002 |
| V$E2F1_Q3 | 191 | 2.001 | 0.000 | 0.002 |
| V$E2F1 _Q6_01 | 192 | 1.998 | 0.002 | 0.002 |
| V$MYC_Q2 | 152 | 1.799 | 0.000 | 0.010 |
| V$MYCMAX_01 | 215 | 1.763 | 0.000 | 0.015 |
| C5 MSigDB Gene Set Collection | | | | |
| Cell_cycle_go_0007049 | 294 | 2.114 | 0.000 | 0.015 |
| Cell_cycle_checkpoint_go_0000075 | 45 | 2.111 | 0.000 | 0.008 |
| Regulation_of _cell_cycle | 170 | 2.108 | 0.000 | 0.006 |
| Cell_cycle_phase | 157 | 2.105 | 0.000 | 0.004 |
| Mitotic_cell_cycle | 140 | 2.098 | 0.000 | 0.004 |
| Dna_replication | 93 | 2.095 | 0.000 | 0.004 |
| Cell_cycle_process | 180 | 2.077 | 0.000 | 0.004 |
| Regulation_of _dna_metabolic_process | 41 | 2.041 | 0.002 | 0.005 |
| G1_stransition_of_mitotic_ cell_ | 27 | 1.971 | 0.002 | 0.006 |
| Regulation_of_cyclin_dependent_protein_kinase_activity | 42 | 1.942 | 0.000 | 0.008 |

The results from the different MSigDB gene set collections were highly coincident and pointed towards a cell cycle deregulation present in 1qGSig tumors:
1.—GSEA using the MSigDB C2 gene set collection found significant enrichment in multiple gene sets from cell cycle studies, highest NES belonging to the “G1 to S cell cycle reactome” gene set.
2.—GSEA using the MSigDB C5 gene set collection, Gene Ontology terms (GO), found significant enrichment in multiple gene sets related to Cell Cycle GOs, mainly cell cycle checkpoints and DNA metabolism.
3.—GSEA using the MSigDB C3 gene set collection (motifs) found significant enrichment in lists of genes known to be transcriptionally regulated by the cell cycle central regulator E2F1.

All of these hits had very low FDR and high NES. FIG. 2c displays one of the most significantly enriched gene sets, corresponding to the gene set “G1 to S cell cycle reactome.”

The possibility that 1q gain CNA-partners might have been the cause of the observed enrichments was discarded by C1 MSigDB gene set collection—GSEA analysis, which found only genes in 1q to be significantly overrepresented due to their genomic position.

Ingenuity Software was used to validate the GSEA results. A gene list from the SAM analysis (q-value<0.02, 1qGSig tumors vs. rest of tumors) was used as the input. Cell cycle and cancer were found to be the most significant functions overrepresented in 1qGSig tumors. Regarding canonical pathways, among others the analysis found DNA metabolism, G1/S Checkpoint Regulation and Protein Ubiquitination Pathway as the main deregulated pathways (FIG. 2d).

### Example 3

Selection of DTL as a Candidate Gene and Real Time RT qPCR Validation

Since the molecular phenotype discovered was up to this point restricted to bioinformatics predictions, it was necessary to empirically prove the ability of 1q gain deregulated genes to exert cell cycle control functions. A decision algorithm for candidate gene selection was created based on several stringent requirements, comprising both biological and numerical criteria. These are the features that made DTL (also known as CDT2, L2DTL or RAMP) the only gene that satisfied these requirements:

1.—It is a gene located in 1q.
2.—It is the most significantly overexpressed gene in 1qGSig-tumors: first gene in the d-value (the most reliable indicator of consistent differential expression) ranked list from the SAM analysis.
3.—DTL fold change in 1q gain tumors (4.78) was quite higher than the expected due to the underlying increase in genomic dosage (1.5).
4.—It is involved in 5 out of 6 of the Ingenuity's predicted deregulated pathways including one, Protein Ubiquitination Pathway, without evident initial link to the pattern of cell cycle deregulation: DTL is a WD40-repeat protein part of the recently discovered CUL4-DDB1^{CDT2} protein-ubiquitin ligase complex, which functions as an adaptor for specific substrate-7 targeting. Jackson, S. & Xiong, Y., Trends Biochem Sci 34, 562-570 (2009). This complex is involved in cell cycle control by regulating the protein levels of multiple G1 to S transition inhibitors (among others p53, p21 and p27) and of factors implicated in DNA replication (CDT1) (Jackson, S. & Xiong, Y., Trends Biochem Sci 34, 562-570 (2009); Higa, L. A., et al., Nat Cell Biol 5, 1008-1015 (2003); Banks, D., et al., Cell Cycle 5, 1719-1729 (2006); Higa, L. A., et al., Cell Cycle 5, 71-77 (2006); Jin, J., et al., Mol Cell 23, 709-721 (2006); Abbas, T., et al., Genes Dev 22, 2496-2506 (2008); Sansam, C. L., et al., Genes Dev 20, 3117-3129 (2006)). Real Time RT-qPCR in a subset of 14 tumor samples confirmed the overexpression of this gene in 1qGSig tumors (FOG 3a).

### Example 4

In Vitro and In Vivo Functional Validation Reveals that DTL is a Potent and Tight Regulator of the G1 to S Transition in ES Cell Lines.

Next, the candidate gene was validated using in vitro and in vivo approaches based on lentiviral shRNA silencing in 1q gain/DTL-overexpressing ES cell lines.

Total RNA from DTL-silenced cell lines was extracted with Trizol (Invitrogen) and hybridized with the GeneChip® Human Genome U133 Plus 2.0 Array (Affymetrix) after quality assessment using an Agilent 2100 Bioanalyzer (Agilent). DNA from the SKES1 cell line was hybridized with the GeneChip® Human Mapping 500K Array Set (Affymetrix).

The MISSION shRNA collection of 5 pLKO.1-shRNA constructions (SIGMA Aldrich) was selected for assays aimed at reducing DTL overexpression. The pLKO.1-non-targeting control construction (pLKO.1-NTC) was included as a control. Constructions were transfected separately in the 293T packaging cell line along with the pMD.G and pCMV-dR8.91 vectors, following a typical lipofectamine 2000 (Invitrogen) protocol. At 48 hours after transfection, viral supernatants were collected, fresh medium added, passed through a 0.44 μm-polysulfonate filter (PALL), polybrene-added to a final 8 μg/ml concentration (SIGMA Aldrich) and used to transduce target ES cell lines.

For assessing the transduction efficiency, TC32 was transduced with the pLKO.1-turboGFP vector and was evaluated by flow cytometry after 48 hours.

For gradual silencing, sh280 and sh2495 supernatants were mixed and progressively diluted with fresh medium before being applied to target cells. After reseeding, cell cycle, apoptosis and proliferation assays were performed at several time points.

For in vivo functional validation of DTL, TC32 and RM82 cell lines were transduced with a progressively diluted DTL-shRNA viral supernatant. Both cell lines were also transduced separately with the pLKO.1-NTC. 48 hours after transduction, cells were trypsinized and suspensions containing 2×10⁶ living cells in a 0.2 ml-final volume composed of RPMI medium and Matrigel Matrix (BD) in a 1:1 proportion, were injected s.c. in one flank (DTL-shRNA transduced cells) or in the other (cells transduced with the pLKO.1-NTC) of five-week old NOD/SCID female mice (Charles River). Twelve mice were used for each cell line (quadruplicates of each silencing condition). The animals were euthanized by anesthetic overdose 19 days after cell injections, and tumors were collected for histopathology analysis, and weights were measured. The study was previously approved by the Bioethics Committee of the CIC-USAL institution.

Five shRNA constructions, each targeting a different region of DTL mRNA, were tested in the RM82 cell line. The shRNA constructions are introduced in Table 3 below.

TABLE 3
Detailed information on the shRNA
constructions used against DTL

| | Sequence of the sh | Exons |
|---|---|---|
| shRNA name | targeting DTL-mRNA | targeted |
| 280_s1c1 | GCTCCCAATATGGAACATGTA/ | Exons 2-3 |
| | TACATGTTCCATATTGGGAGC | |
| | (SEQ ID NO: 5) | |
| 424_s1c1 | CCTGGTGAACTTAAACTTGTT/ | Exons 4-5 |
| | AACAAGTTTAAGTTCACCAGG | |
| | (SEQ ID NO: 6) | |
| 425_s1c1 | CTGGTGAACTTAAACTTGTTA/ | Exons 4-5 |
| | TAACAAGTTTAAGTTCACCAG | |
| | (SEQ ID NO: 7) | |
| 1358_s1c1 | GCCTAGTAACAGTAACGAGTA/ | Exon 14 |
| | TACTCGTTACTGTTACTAGGC | |
| | (SEQ ID NO: 8) | |
| 2495_s1c1 | CCGAGGATGAATGCTGTGTTT/ | 3′-UTR |
| | AAACACAGCATTCATCCTCGG | |
| | (SEQ ID NO: 9) | |

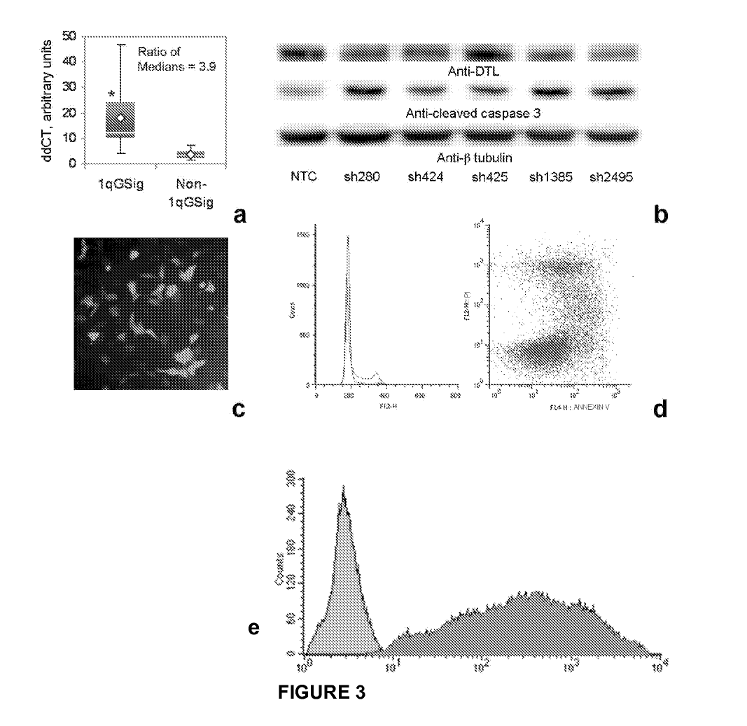

All of them decreased DTL protein levels with different efficiencies (FIG. 3b). Moreover, DTL silencing confirmed this genes ability to regulate the cell cycle progression since its abrogation induced a G0/G1-cell cycle arrest (FIG. 3d). It also induced an increase in apoptosis (FIG. 3b, anti-cleaved caspase-3 blot, and FIG. 3d). A pLKO.1-turboGFP construction was used to ensure that the transduction of the whole population had been achieved, using both fluorescence microscopy (FIG. 3c) and flow cytometry (FIG. 3e). The two shRNA constructions (sh280 and sh2495) inducing the strongest degree of silencing and covering the most complete range of mRNA isoforms were selected. Further validations were performed in two cell lines (RM82 and TC32) with 1q gain and the highest DTL-mRNA levels. We opted for a strategy of gradual silencing, accomplished by progressively diluting the viral supernatant used for transduction. With this approach, DTL protein levels were never abrogated (which was observed to arrest in G0/G1 around a 90% of the cell population and to induce massive apoptosis (see, e.g., FIG. 3d).

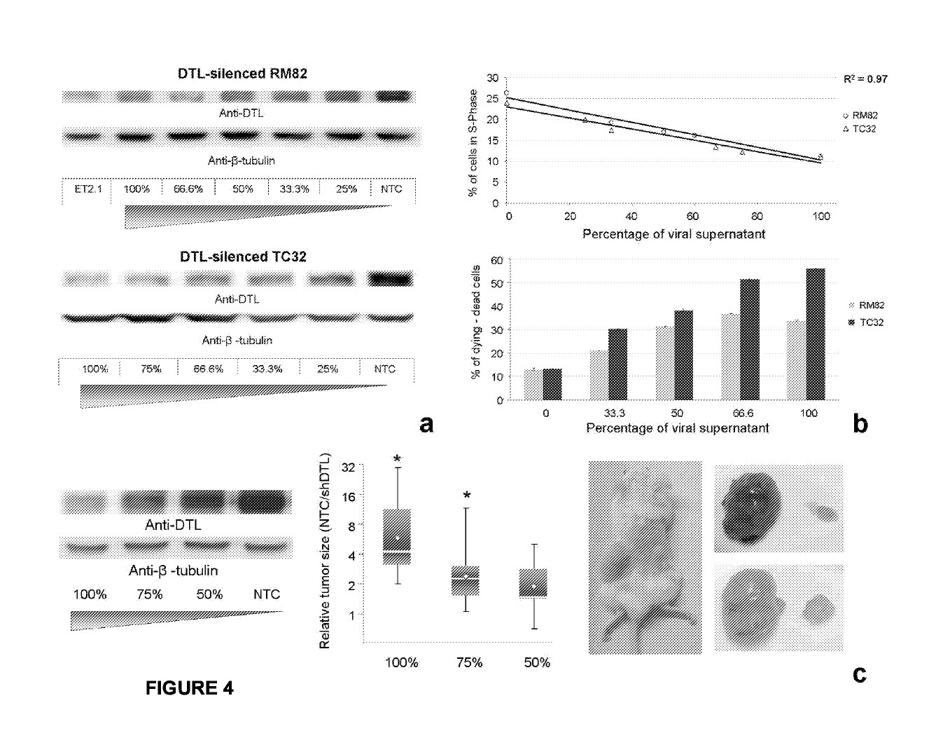

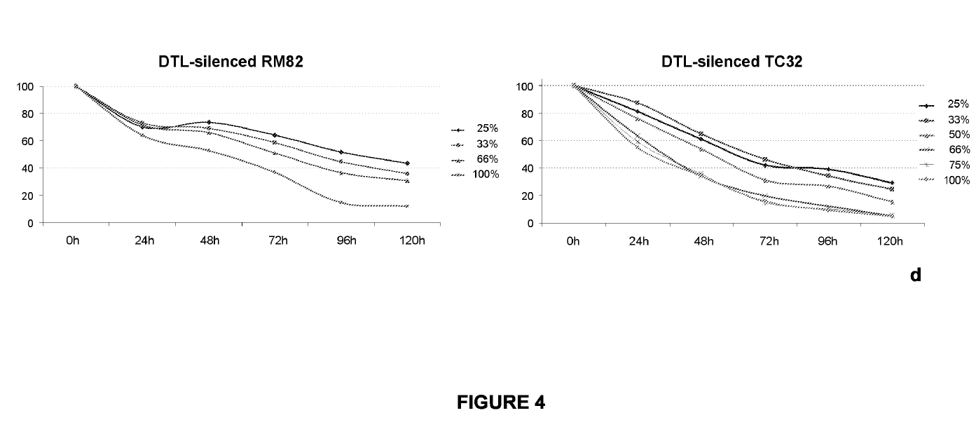

Instead, overexpression was reduced according to several degrees of silencing (FIG. 4a), allowing functional studies to be carried out.

Cells silenced by this approach were next subjected to cell cycle, apoptosis and proliferation assays. The results revealed a tight regulation of the cell cycle exerted by DTL in both cell lines, insofar that the degree of silencing (expressed as the percentage of the viral supernatant used in each silencing-point) and the corresponding percentage of the cell population in S-phase fitted in a linear regression (FIG. 4b, top panel), indicating these cell lines dependency on DTL for S-phase entry. As expected, proliferation also decreased in parallel to the degree of silencing (see, FIG. 4d. The induced apoptosis was also gradual (FIG. 4b, bottom panel).

The in vitro results suggest that other G1 to S transition regulators, not yet described, might be under the control of the CUL4/DDB^{CDT2} complex. This model is consistent with the higher proliferation rates that we have detected in 1q gain ES tumors.

In a similar manner, DTL-silenced RM82 and TC32 cell lines were injected subcutaneously in mice for in vivo studies. The range of silencing used reached only 50% of undiluted supernatant in order to prevent the overgrowth of small untransduced populations, seeing that the extension of the in vivo assay is substantially longer than the in vitro one. The results confirmed the progressive effects obtained in vitro although differences found were milder (FIG. 4c). The most silenced condition had dramatic effects in vivo, achieving an almost complete suppression of tumor growth in several of the animals (FIG. 4c, right panel).

In conclusion, results from in vitro and in vivo functional validations suggest that DTL, the most significantly overexpressed gene in 1q-gain tumors (1qGT), is a major actor in this deregulation since it demonstrated a tight control over cell cycle through at least p53, p21 and p27, and was a principal contributor to the 1q gain-expression profile. Moreover, 1qGT showed higher proliferation rates.

### Example 5

### DTL Silencing Induces Dramatic Changes in the Protein Levels of Several G1-S Transition Regulators in ES Cell Lines.

The RM82, TC32, and STAET10 (which also bears 1q gain and medium to high expression of DTL cell lines were subjected to a blot-screening aimed at identifying specific changes in protein levels induced by the silencing of DTL. Several antibodies against G1 to S regulators were selected for this part of the study.

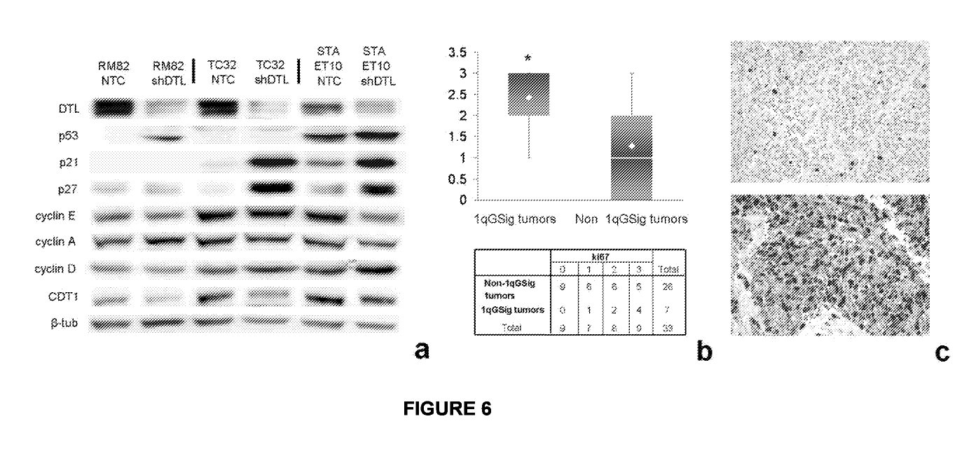

Full-silencing of DTL resulted in dramatic increases of p21 and p27 and marked increases in p53, confirming the reported regulatory effect of the CUL4/DDB1^{CDT2} complex on these proteins (Banks, D., et al., Cell Cycle 5, 1719-1729 (2006); Higa, L. A., et al., Cell Cycle 5, 71-77 (2006); Abbas, T., et al., Genes Dev 22, 2496-2506 (2008); Kim, Y, et al., Genes Dev 22, 2507-2519 (2008); Nishitani, H., et al., J Biol Chem 283, 29045-29052 (2008)) (FIG. 6a). Strikingly, as shown above, RM82, a cell line lacking both p21 and p27, was also deeply affected by DTL-silencing suggesting that other regulators of the G1 to S transition not yet described might be under the control of this protein-ubiquitin ligase complex. Overall, these findings highlight the pleiotropic regulation of the cell cycle exerted by DTL in ES cell lines.

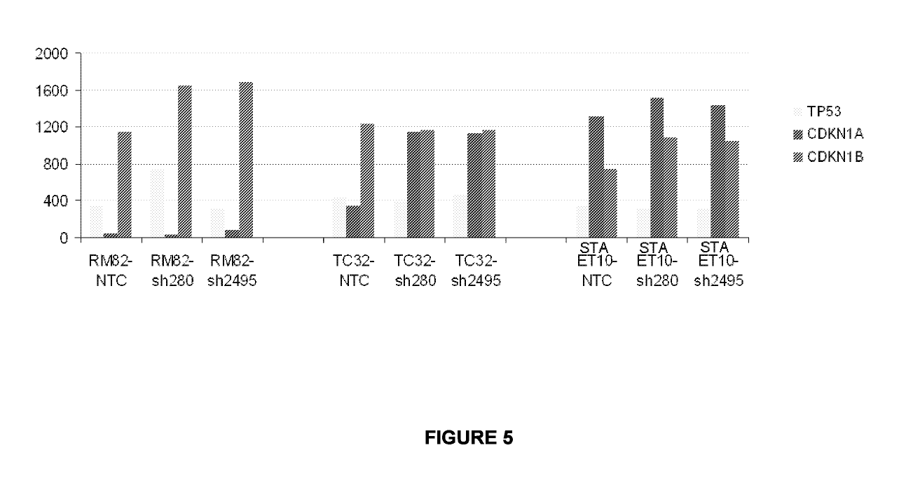

As expected, the changes detected with this blot screening were caused by post-transcriptional processes since the levels of CDKN1A, CDKN1B or TP53 probesets from the expression microarrays (see below) showed no meaningful variations (see, FIG. 5). The only change detected, belonging to the CDKN2b (p27^{KIP1}) probeset in TC32, a three-time increase, is not enough to explain the change in protein levels.

### Example 6

The High Concordance Between the 1qGSig-Related and the DTL-Induced Expression Profiles Suggests that DTL is a Major Contributor to the 1q Gain Profile.

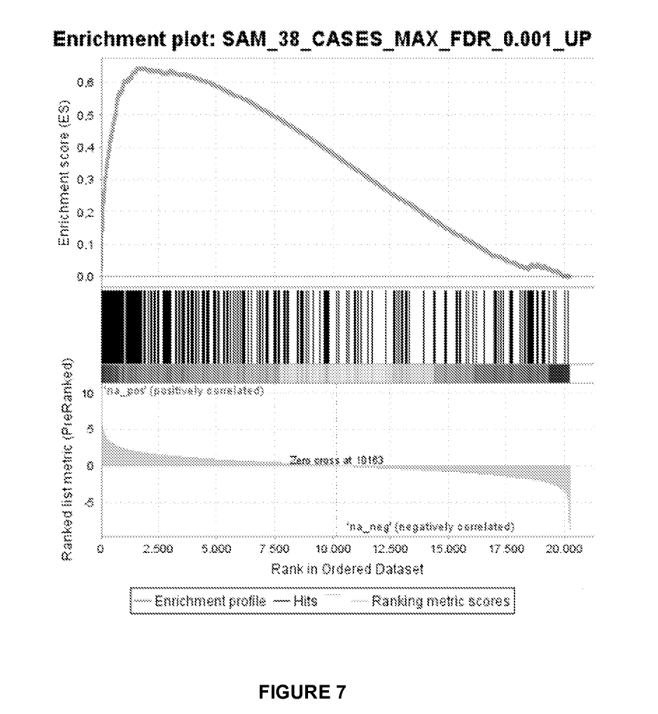

Expression microarray studies were performed using RNA from the DTL-silenced ES cell lines (RM82, TC32 and STAET10, silenced using both sh280 and sh2495 transduced separately) in order to obtain a DTL-induced expression profile. This profile was compared with the SAM of the tumors grouped according to the 1qGSig, using GSEA (see, FIG. 7). A high concordance between both profiles was found as reflected by the high NES (3.16) obtained. This result reveals a significant contribution of DTL to the transcriptomic pattern associated with the 1q gain.

### Example 7

1qGSig ES Tumors have Higher Proliferation Rates.

According to the bioinformatics and the in vitro results, 1q gain ES was expected to have a higher proliferation rate. In order to validate this assumption, a tissue microarray (TMA) composed of 33 samples from the 38-tumor set was used for the immunohistochemical analysis (IHC) of Ki-67, a well-established marker of proliferation, and was scored with 4 values depending on the percentage of immunostained cells. As a result, 1qGSig ES tumors were confirmed to have significantly higher Ki-67-proliferation indexes (FIG. 6b, Kendal-Tau <0.01). Examples of a negative and a positive tumor (scored 3) for Ki-67 IHC can be seen in FIG. 6c.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

What is claimed is:

1. A method of identifying in the neoplasm(s) from a subject any of the following molecular features with prognostic value, said method comprising:
(a) obtaining a neoplastic sample from said subject; and
(b) (i) screening said neoplastic sample for genomic copy number alterations (CNA) and/or the expression levels of the 1qGSig genes, wherein neoplastic samples having 1q gain and/or the 1qGSig are statistically associated with poor prognosis for said subject;
(ii) screening said neoplastic sample for genomic copy number alterations (CNA), wherein neoplastic samples not harbouring CNA are statistically associated with good prognosis for said subject;
(iii) measuring DTL expression levels (RNA and protein) in said neoplastic sample, wherein neoplastic samples having (i) a 1q gain and/or (ii) statistically higher amounts of DTL relative to a control are statistically associated with poor prognosis for said subject; or
(iv) any combination of (i), (ii) and (iii).

2. The method of claim 1, wherein the neoplasm is selected from the group consisting of Ewing sarcoma, lung cancer, prostate cancer, colorectal cancer, thyroid cancer, renal cancer, adrenal cancer, pancreatic cancer, myeloma, Wilms tumor, medulloblastoma, neuroblastoma, hepatic carcinoma, breast cancer and central and peripheral nervous system cancer.

3. The method of claim 1, wherein the neoplasm is Ewing sarcoma.

4. The method of claim 1, wherein the adverse prognoses include relapse or poor survival for the subject.

5. The method of claim 1, wherein the good prognosis includes absence of relapse or a disease-free survival for the subject.

6. The method of claim 1, comprising steps (b)(i) or (b)(ii), wherein the screening is performed using array CGH or any technique able to identify CNA in a sample.

7. The method of claim 1, comprising steps (b)(i) or (b)(iii), wherein the screening is performed by measuring the expression levels of the genes from the 1qGSig, or a part of them, and/or the expression levels of DTL, by Real Time (RT) qPCR, Western Blot, immunohistochemistry, or any other technique able to evaluate gene expression levels.

8. A method of inhibiting tumor growth in a subject diagnosed with a neoplasm displaying overexpressed DTL protein levels, said method comprising inhibiting the expression of a DTL gene by introducing an interfering nucleotide sequence that targets at least a fragment of a polynucleotide sequence of the DTL gene and silences the gene.

9. The method of claim 8, wherein the neoplasm is Ewing Sarcoma.

10. The method of claim 8, wherein the interfering nucleotide sequence comprises shRNA constructions.

11. A method of inhibiting tumor growth in a subject diagnosed with a neoplasm displaying overexpressed DTL protein levels, said method comprising administering a silencing oligonucleotide (siRNA) or plasmid construction containing such oligonucleotide (shRNA)

12. The method of claim 11, wherein the administration to said patient is selected from the group consisting of subcutaneous, orally, vaginally, nasally, intravenously, intramuscularly, intraperitoneally, transdermally or transmucosally.

13. The method of claim 11, wherein the neoplasm is Ewing Sarcoma.
